(19) United States
(12) Patent Application Publication
(10) Pub. No.: US 2006/0234244 A1
(43) Pub. Date: Oct. 19, 2006

(54) System for analyzing bio chips using gene ontology and a method thereof

(76) Inventors: Yang-Suk Kim, Gyeonggi-do (KR); Jung-Uk Hur, Mungyeong-si (KR); Sung-Geun Lee, Busan (KR)

Correspondence Address: FLYNN THIEL BOUTELL & TANIS, P.C., 2026 RAMBLING ROAD, KALAMAZOO, MI, 49008-1631, US

(21) Appl. No.: 10/579,504

(22) PCT Filed: Aug. 23, 2004
(86) PCT No.: PCT/KR04/02117

§ 371 Date: May 15, 2006

(30) Foreign Application Priority Data
Aug. 30, 2003 (KR) 10-2003-0060528

(51) Int. Cl. C12Q 1/68; G06F 19/00

(52) U.S. Cl. 435/6; 702/20

(57) ABSTRACT

Disclosed is a system for analyzing a bio chip using Gene Ontology(hereinafter referred to “GO”) and a method thereof. According to a preferred embodiment of the present invention, it is provided a system for analyzing a bio chip comprising: a GO(gene ontology) term assigning part for receiving a statistical clustering data obtained from empirical results of the bio chip, and assigning relevant GO terms to every gene contained in each cluster; a GO code converting part for converting the GO terms assigned by the GO term assigning part to the genes into GO codes, the GO code comprising a group of predetermined numbers; and a biological meaning extracting part for calculating pseudo distances between one of GO terms on GO tree structure contained in a predetermined group and the GO terms corresponding to the genes contained in the cluster, and calculating at least one of average pseudo distance or maximum pseudo distance of the calculated pseudo distances, and calculating at least one of average pseudo distances or maximum pseudo distances for all GO terms included on GO tree structure in the predetermined group and the GO terms corresponding to the genes contained in the cluster, and determining an optimum GO term matching with the cluster.

### BACKGROUND OF THE INVENTION

1. Technical Field

The present invention relates to a system for analyzing a bio chip using a Gene Ontology(hereinafter referred to “GO”) and a method thereof, more particularly to a system for biologically analyzing an expression pattern of gene obtained from an experiment of a DNA chip or a Microarray by means of modeling of GO hierarchical structure and to a method thereof.

2. Background Art

Since Watson and Crick discovered double helix structure of DNA molecule, there has been rapid progress in the field of biology. After the discovery a restriction enzyme was discovered, a hybridization technique was developed, and a PCR(polymerase chain reaction) was developed. These developments and discoveries helped us understand a biological characteristic in the molecular level. However, as a need for experiment such as Human Genomic Project(HGB) where the biological characteristic is not fragmentarily but wholly understood increases, various studies for discovering function of nucleotide sequence have been conducted and devices were developed such as DNA chips. In addition, various researches associated with Bioinformatics and Functional Genomics are being actively performed so as to effectively employ data obtained from the HGP or the DNA chip.

Generally, bio chips are classified into a Microarray and a Microfluidics chip. Thousands or ten thousands of DNA or protein at regular intervals are arrayed in the Microarray including DNA chip and protein chip. Until now, the Microarray has been broadly used as a bio chip. The Microfluidics chip is used to analyze a reaction pattern of a bio molecule or a sensor arrayed in the chip and the sample flowing in the chip.

Target DNA, cDNA or oligonucleotide is attached onto a surface such as glass surface, nitrocellulose membrane and silicon in the DNA chip. In other words, in the DNA chip, cDNA whose nucleotide sequence is known or oligonucleotide probe is micro-arrayed on the small solid surface.

The DNA chip has the sample interacting with the probe marked with a fluorescent material or a radioactive isotope, and it may be employed in a identification of a gene expression intensity and a mutation, a single nucleotide polymorphism(SNP), a diagnosis of diseases, and high-throughput screening(HTS). If DNA fragments of a sample to be analyzed is associated with the probes in the DNA chip, the fragments and the probes arrayed in the DNA chip form a hybrid state according to the complementary nucleotide sequences of the fragments and the probes. By means of observing and interpreting the hybrid state through optical method and chemical method, the nucleotide sequences of a sample DNA may be found out. Accordingly, expression information of many genes can be known simply and quickly through the DNA chip. At present, the DNA chip is used for the development of new drug and diagnosis of a disease.

Analysis of DNA chip has been carried out by a statistical method and a biological method.

Numerical gene expression intensity is obtained by image analysis and the clusters showing similar expression patterns are grouped by clustering techniques.

As the clusters are grouped only by the statistical method, for identifying biological meaning thereof, general biological meanings are granted to the clusters and the credibility of the clusters are biologically identified using known functions about each gene contained in the clusters.

A conventional method for biologically granting the general meaning to the clusters comprises the methods for extracting functions of genes from the literature or biological information database and comparing with them. At this time, such biological database information includes fundamental DNA information of NCBI(National Center for Biotechnology Information) functional category information of MIPS(Munich Information Center for Protein Sequence) or CGAP(Cancer Genome Anatomy Project) and protein information of Swiss-Prot, and the like.

However, common problems with the conventional method as described above are that the method was manually conducted and was difficult to automatically analyze the meaning of a cluster due to the diversities of biological terms.

In case of conventional biological database, the Swiss-Prot employed as information source of proteins classifies the functions of proteins well by using key-words, however, uniform correlation or hierarchy between the key-words does not exist and hence it was difficult to automatically analyze DNA chip data for biological meanings.

Furthermore, group information about specific field such as CGAP(Cancer Genome Anatomy Project) is applied only to the corresponding field and is not specific because too broad function is dealt with.

Accordingly, the conventional method may require much time to grant a biological meaning to the cluster extracted only by a statistical method and could not grant a detailed and correct biological meaning thereto.

Meanwhile, GO Consortium provides GO terms, which refers to an organization of biological terms and vocabularies classified. The GO Consortium is constituted in order to integrate the biological terms and provides integrated terms which may be commonly employed to explain the function of genes in all biological species. In present, GO terms comprise about over ten thousand terms. Ultimately, GO refers to a study of hierarchy between genes or key-words implied in the genes and is employed in bioinformatics.

These GO terms have characteristics that each term has a tree-like structure of hierarchy and every term is classified into one of three categories. That is, about ten thousand terms which are classified into three categories have a hierarchy similar to the tree structure. The GO terms are divided into three categories such as i) molecular function, ii) biological process and iii) cellular component and grant classical controlled vocabulary to each category to analyze biological meaning of DNA chip. The categories are not exclusive each other and they are divided in order to describe one gene more effectively.

The present invention relates to a system for automatically granting biological meanings to a cluster by using these GO terms and a method thereof

### SUMMARY OF THE INVENTION

Therefore, the present invention has been developed to solve the above-mentioned problems. An object of the present invention is to provide a system for analyzing a bio chip by using the GO such that a biological analysis on genes expression patterns of a DNA chip data may be performed systematically through modeling of GO hierarchical structure and a method thereof.

Another object of the present invention is to provide a method for extracting most common and ideal function of genes which belong to the cluster formed through a statistic clustering of a data obtained from the DNA chip by using the GO terms and the tree structure.

To accomplish the above-described objects, according to an embodiment of the present invention, it is provided to a system for analyzing a bio chip comprising:

a GO(gene ontology) term assigning part for receiving a statistical clustering data obtained from empirical results of the bio chip, and assigning relevant GO terms to every gene contained in each cluster;

a GO code converting part for converting the GO terms assigned by the GO term assigning part to the genes into GO codes, the GO code comprising a group of predetermined numbers; and

a biological meaning extracting part for calculating pseudo distances between one of GO terms in a predetermined group on GO tree structure contained and the GO terms corresponding to the genes contained in the cluster, and calculating at least one of average pseudo distance or maximum pseudo distance of the calculated pseudo distances, and calculating at least one of average pseudo distances or maximum pseudo distances for all GO terms included in the predetermined group on GO tree structure and the GO terms corresponding to the genes contained in the cluster, and determining an optimum GO term matching with the cluster.

The GO term assigning part may assign GO terms to the genes using biology database mining.

The GO code converting part may covert the GO terms into the GO codes according to a level of a GO term, a parent-node of the GO term and an order of the GO term in the level.

The biological meaning extracting part comprises:

an optimum cross-point extracting part for extracting optimum cross-points between the GO terms on the GO tree structure and the GO terms assigned to the genes contained in the predetermined group;

a pseudo distance calculating part for calculating pseudo distances between the GO terms on the GO tree structure and the GO terms assigned to the genes contained in the cluster by using the optimum cross-points information;

an average pseudo distance calculating part for calculating average pseudo distance of the pseudo distances calculated from the pseudo distance calculating part;

a maximum pseudo distance determining part for determining maximum distance among the pseudo distances calculated from the pseudo distance calculating part; and

an optimum matching node determining part for comparing average pseudo distances or maximum pseudo distances for all GO terms contained in the predetermined group, and determining a GO term with minimum value of the average pseudo distance or of the maximum pseudo distance to be optimum matching node of the cluster.

The GO terms contained in the predetermined group may be all terms on the GO tree structure.

The GO terms contained in the predetermined group may be GO terms included in a selected level on the GO tree structure.

The optimum cross-point extracting part may determine a GO term in the lowest level among GO terms which include two GO terms in lower level on the GO tree structure to be the optimum cross-point.

The GO tree structure may comprise a level which a predetermined weight is granted to, and wherein the pseudo distance calculated by the pseudo distance calculating part is the weight granted to a level where the optimum cross-point exists.

Meanwhile, according to another embodiment of the present invention, it is provided to a method for analyzing a bio chip comprising:

a) receiving a statistical clustering data obtained from empirical results of the bio chip to assign relevant GO terms to every gene contained in each cluster;

b) converting the GO terms assigned to the genes into GO codes, the GO code comprising a group of predetermined numbers;

c) calculating pseudo distances between one of GO terms contained in the predetermined group on GO tree structure and the GO terms corresponding to the genes contained in the cluster by using the GO codes;

d) calculating at least one of average pseudo distance or maximum pseudo distance of the pseudo distances calculated in the step (c); and

e) repeating the step (c) and the step (d) for every GO term on the GO tree structure contained in the predetermined group to determine an optimum GO term matching with the cluster.

Meanwhile, according to another embodiment of the present invention, it is provided to a digital device readable medium containing program instructions for executing an analysis of a bio chip, the medium comprising the program instructions for:

a) receiving a statistical clustering data obtained from empirical results of the bio chip, and for assigning relevant GO terms to every gene contained in each cluster;

b) converting the GO terms assigned to the genes into GO codes, the GO code comprising a group of predetermined numbers;

c) calculating pseudo distances between one of GO terms on GO tree structure contained in a predetermined group and the GO terms corresponding to the genes contained in the cluster by using the GO codes;

d) calculating at least one of average pseudo distance or maximum pseudo distance of the pseudo distances calculated in the step (c); and

e) repeating the step (c) and the step (d) for every GO term on the GO tree structure contained in the predetermined group to determine an optimum GO term matching with the cluster.

### DISCLOSURE OF THE INVENTION

Hereinafter, a system for analyzing the DNA chip by using GO and a method thereof according to a preferred embodiment of the present invention will be described in more detail with reference to the accompanying drawing.

FIG. 1a illustrates an example of GO structure and FIG. 1b illustrates an example of GO text structure.

Prior to description of the present invention, a hierarchical structure of the GO will be described. As shown in FIG. 1a, on the hierarchical structure the highest(the first) level corresponds to top GO category, the second level corresponds to the three categories of GO, i.e. molecular function(MF), biological process(BP) and cellular component(CP), and trees for lower level such as the third, the fourth and the fifth level are formed. As the level is lower, function of a GO term becomes more detailed and specific.

As shown in FIG. 1a, the GO structure is not a perfect tree structure but a directed cycle-free graph structure. In the present invention directed graph GO structure is converted into tree structure, and the converted structure is employed. Since a method for converting a directed graph structure into a tree structure is simple and is already known to those skilled in the art, the detailed method will be not described here. FIG. 1b illustrates text GO structure which is converted from the tree structure, GO term in lower level is recorded in a row indented to the right side than GO terms in higher level and GO terms in the same level are recorded with the same indentation. The text GO model may be obtained from the GO consortium.

FIG. 2 is a block diagram of a system for analyzing a DNA chip using GO according to a preferred embodiment of the present invention.

As shown in FIG. 2, a system for analyzing a DNA chip according to an embodiment of the present invention may include a clustering part(200), a GO term assigning part(202), a GO code converting part(204), a GO code storing part(206) and a biological meaning extracting part(208).

The clustering part(200) performs clustering of genes showing similar expression patterns by using the expression intensity data of the DNA chip. The expression intensity of a DNA chip is obtained under various conditions, the clustering is a process that divides the genes showing similar expression patterns into groups among a plurality of genes contained in the DNA chip. Accordingly, a plurality of clusters may be formed as a result of the clustering, each cluster includes a plurality of genes showing similar expression patterns. Since various algorithms on the clustering are known to those skilled of in the art, a detailed clustering method will not be described here, and the conventional clustering algorithms may be applied to the present invention.

The GO terms assigning part(202) assigns relevant GO terms to each gene contained in a cluster after the clustering is performed. It determines which terms of function defined in the GO corresponds to the genes contained in the cluster and assigns the GO terms to each gene. When a gene exhibits a plurality of function, a plurality of GO terms may be assigned to the gene.

According to a preferred embodiment of the present invention, GO terms associated with a specific gene may be obtained from biology database through the internet. The biology database accessible through the internet may include Unigene, LocusLink, Swiss-Prot and MGI, etc. Most of the above databases provide the GO terms associated with the function of the genes. Though relevant GO terms are not offered directly by the database, they may be obtained from function information of the genes offered thereby. The UniGene offers the gene information of DNA level provided by NCBI(National Center for Biotechnology Information), LocusLink offers function of each genes and a sequence information having reference as a result of Reference Sequence Project of the NCBI, Swiss-Prot offers information of protein level provided by Swiss Institute of Bioinformatics, and MGI offers DNA information of mouse.

According to another embodiment of the present invention, in addition to the above databases accessible through the internet, self-constructed databases and files may be employed to assign GO terms to the genes.

The GO code converting part(204) converts the GO terms assigned to the genes into predetermined GO codes. Since the GO terms are characters, it is difficult to determine distance between a GO term assigned to a gene and another GO terms on the GO tree structure. Accordingly, the present invention converts a GO term into a combination of predetermined numbers. As the GO term is converted into the combination of numbers, it is possible to numerically calculate the distance between a GO code of a specific node(GO term) and a GO code of another node on the tree structure.

A detailed constitution of the GO code and method for converting a GO term into a GO code will be described referring to another figures.

The GO code storing part(206) stores information on GO codes which are previously converted from GO terms on tree structure, the GO code converting part(204) may convert the GO terms into the GO codes by using the above information stored at the GO code storing part(206).

The biological meaning extracting part(208) determines the biological meanings of a cluster, which is a group of genes showing similar expression patterns. The biological meaning extracting part(208) may determine which GO terms on GO tree structure is the closest to the common function of the genes contained in the cluster, and may determine representative function of the genes contained in the cluster by associating the closest GO term with the cluster.

As described above, since the clustering is performed by a statistical method without considering the biological meaning, it took a long time to grant the biological meaning to the cluster. However, according to the present invention, because a GO term which is the closest to the cluster is previously determined by a program, time for analyzing the biological meaning about the cluster may be remarkably reduced.

To determine a GO term that is the closest to the meaning of a cluster, the biological meaning extracting part(208) calculates a degree of intimacy(closeness) between a node on the GO tree structure and each gene contained in the cluster. To calculate the degree of intimacy, the present invention suggests a concept named Pseudo Distance. A method for calculating the pseudo distance will be described in detail later.

The biological meaning extracting part(208) calculates pseudo distances between a node on the GO tree structure and the genes contained in the cluster and then calculates average pseudo distance or maximum pseudo distance between the node on the GO tree structure and every gene contained in the cluster.

The above described process, which calculates the average pseudo distance or the maximum pseudo distance between the node on the GO tree structure and every gene contained in the cluster, may be performed for all nodes on the GO tree structure or some nodes selected by user. A node(GO term) on the GO tree structure, which corresponds to the minimum value of the average pseudo distances or of the maximum pseudo distances, may be determined to be the closest node to the cluster. The biological meaning of the cluster may be determined to be the GO term corresponding to the node.

FIG. 3 is a drawing for explaining an exemplary process that converts a GO term into a GO code.

A GO term is converted to a GO code depending on the level of the GO term on the GO tree structure and an order in the level.

In FIG. 3, GO term 300, which belongs to the first level, is the first node in the first level. At this time, the GO term 300 is converted to a GO code, “10000000000000”. The GO code has fifteen figures because the GO level comprises fifteen level, the first figure of the GO code represents first level, the second figure represents second level, and the like. Since the GO term 300 is the first GO term in the first level, the first figure of the GO code of the GO term 300 represents “1” and the rest of the figures of the GO code represent zero. A GO term 302 belongs to the second level and is the lower node of the GO term 300. At this time, the GO term 302 is converted to a GO code, “110000000000000”.

Since the GO term 302 belongs to the second level, its GO code has zero value from the third figure to the fifth. Further, since the GO term 302 is a son-node of the GO term 300, the first figure of the GO term 302 is equal to that of the GO term 300. Furthermore, since the GO term 302 is the first node in the second level which is the lower level of the GO term 300, the second figure of the GO code of the GO term 302 represents “1”.

By the same method, a GO term 304 may be converted into a GO code, “120000000000000”.

The GO term 310 which belongs to the third level, is a son node of the GO term 302 and is the second node among son nodes of the GO term 302. Accordingly, the GO term 310 may be converted into a GO code, “112000000000000”. Likewise, a GO term 312 may be converted into a GO code, “121000000000000”.

Since a GO term is converted into a GO code through the above method, the GO code includes information on the level of the GO term and the parent-node of the GO term.

FIG. 4 is a block diagram showing a detailed constitution of the biological meaning extracting part according to a preferred embodiment of the present invention.

As shown in FIG. 4, the biological meaning extracting part according to an embodiment of the present invention may include an optimum cross-point extracting part(400), a pseudo distance calculating part(402), an average pseudo distance calculating part(404), a maximum pseudo distance determining part(406) and an optimum matching node determining part(408).

The optimum cross-point extracting part(400) extracts an optimum cross-point between two nodes in order to calculate the pseudo distance. The cross-point extracting step is a prior step of calculating the pseudo distance, and the cross-point between two nodes refers to a node that belongs to the lowest level among high level nodes which include both of the two nodes on the GO tree structure.

For example, referring to FIG. 3, there are the GO term 300 and 302 in higher nodes including both the GO term 308 and 310. Since the GO term 302 is lower node than GO term 300, GO term 300 is the optimum cross-point between the GO term 308 and 310.

By using the GO code, the optimum cross-point can be easily obtained. In FIG. 3, a GO code of the GO term 308 is “111000000000000” and a GO code of the GO term 310 is “112000000000000”. Since the above two GO codes have the same value up to the second figure, an optimum cross-point between the GO term 308 and the GO term 310 exists in the second level and is the first node(as the second figure is 1) of son-nodes of a first node(as the first figure is 1) in the first level.

The pseudo distance calculating part(402) calculates a pseudo distance between two nodes on the GO tree structure by using the above optimum cross-point information. As described above, the pseudo distance calculating part(402) calculates pseudo distance between a specific GO term(node) on the GO tree structure and the GO terms(nodes) assigned to each genes contained in the cluster. Calculation of the pseudo distance is performed for all nodes on the GO tree structure or some nodes selected by user.

According to an embodiment of the present invention, a predetermined weight is granted to each level of the GO tree structure and the pseudo distance may be defined as an weight of a level including an optimum cross-point between two GO terms(nodes). If the two nodes are the same, the pseudo distance is defined as zero.

FIG. 5 is a drawing showing an exemplary process that calculates a pseudo distance between two nodes on GO tree structure.

As shown in FIG. 5, a numerical weight is granted to each level of the GO tree structure(1 level-150, 2 level-140). In FIG. 5, an optimum cross-point between a node 500 and a node 502 is a node 504. The node 504 exists in the third level, an weight granted to the third level is 130. Accordingly, a pseudo distance between the node 500 and 502 is 130.

The average pseudo distance calculating part(404) calculates the arithmetic average of the pseudo distances after the pseudo distances between a specific GO term(node) on the GO tree structure and the GO terms assigned to each gene contained in one cluster have been calculated by the pseudo distance calculating part. The calculated average pseudo distance is used as a barometer representing a degree of association between a specific node on the GO tree structure and a cluster.

The maximum pseudo distance determining part(406) extracts a maximum of the pseudo distances after the pseudo distances between a specific GO term(node) on the GO tree structure and the GO terms assigned to every gene contained in one cluster have been calculated by the pseudo distance calculating part. The larger the maximum of the pseudo distances is, the higher is a possibility that the cluster includes bad genes impairing a general consensus of genes which belong to the cluster. The cluster is a group of genes showing similar expression pattern gathered by a mathematical method, and therefore, biological consensus is not considered enough. Accordingly, the biological consensus of genes contained in the cluster can be determined by calculating maximum pseudo distance. The optimum matching node determining part(408) determines a node of which the average pseudo distance and maximum pseudo distance is the minimum and then determines the node as an optimum matching node of the cluster. Accordingly, a GO term corresponding to the node is a representative term, a biological meaning may be assigned to the cluster obtained from a statistical method. The nodes having the minimum value of the average pseudo distance and the maximum pseudo distance may be the same or not. At this case, the optimum matching node determining part(408) may determine an optimum matching node by using one of the minimum value of the average pseudo distance or of the maximum pseudo distance.

FIG. 6 is a flow chart of analyzing a DNA chip by using GO according to a preferred embodiment of the present invention.

As shown in FIG. 6, the method according to the present invention may include the steps of receiving a statistical clustering data obtained from the empirical results of the bio chip(S10), assigning GO terms to the genes contained in each cluster(S20), converting the GO terms assigned to the genes by the GO term assigning part into GO codes(S30), calculating pseudo distances between one of GO terms on GO tree structure and the GO terms corresponding to the genes contained in the cluster by using the converted GO codes(S40), calculating average pseudo distance of the pseudo distances calculated in the step S40(S50), calculating maximum pseudo distance of the pseudo distances calculated in the step S40(S60); and calculating average pseudo distances and maximum pseudo distances of the cluster for every GO term on the GO tree structure(S70), associating the node having a minimum value of the average pseudo distances or the maximum pseudo distances with the cluster and extracting a biological meaning of the cluster(S80).

Referring to FIG. 6, a method for biologically analyzing an expression pattern of a gene obtained from the DNA chip by using the GO structure will be described in the following.

Firstly, a process for assigning GO terms to each gene contained in the cluster obtained from a statistical clustering method and converting the assigned GO terms into GO codes is performed.

In more detail, after receiving the clustering data(S10), the GO terms corresponding to each gene are obtained through a database mining and the obtained GO terms are assigned to the genes(S20). At this time, using a file where GO terms are assigned through the database mining, the GO terms may be assigned to the genes contained in the cluster. Then, the GO terms assigned to the genes in the cluster are converted into GO codes using a GO code file which includes GO code information for all GO terms on GO tree structure(S30).

After the GO terms are converted into the GO codes, pseudo distances between a specific node on the GO tree structure and the GO terms(nodes) assigned to the genes contained in the cluster are calculated(S40). As described above, the optimum cross-point is extracted in order to calculate the pseudo distance between two nodes, and the weight of the level including the extracted optimum cross-point is determined to be the pseudo distance.

After pseudo distances between the specific node on the GO tree structure and the GO terms(nodes) assigned to the genes contained in the cluster are calculated, an average value of the calculated pseudo distances is calculated(S50) and an maximum value of the calculated pseudo distances is obtained(S60).

The process which calculates the pseudo distances between the specific node on the GO tree structure and the GO terms(nodes) assigned to the genes contained in the cluster is performed for all nodes on the GO tree structure. At this time, a GO node having minimum value of the average pseudo distances and the maximum pseudo distances is determined to be an optimum matching node of the cluster and the GO term corresponding to the GO node is determined to be biological function of the cluster(S80). It would be obvious to those skilled in the art that only one of the GO nodes having a minimum value of the average pseudo distances or the maximum pseudo distances may be employed in order to determine the optimum matching node.

According to another embodiment of the present invention, average pseudo distances may not be calculated for all nodes on the GO tree structure but for some nodes in a specific level selected by a user. At this case, one of the GO terms in the specific level selected by the user may be determined to be a biological meaning of the cluster. When a level is previously indicated, the biological meaning may be easily extracted in a lower level where the biological meaning is difficult to find out comparatively.

### BRIEF DESCRIPTION OF THE DRAWINGS

FIG. 1a illustrates an example of GO structure, FIG. 1b illustrates an example of GO of text structure.

FIG. 2 is a block diagram of a system for analyzing a DNA chip using GO according to a preferred embodiment of the present invention.

FIG. 3 is a drawing for explaining an exemplary process that converts a GO term into a GO code.

FIG. 4 is a block diagram showing a detailed constitution of a biological meaning extracting part according to a preferred embodiment of the present invention.

FIG. 5 is a drawing showing an exemplary process that calculates a pseudo distance between two nodes on the GO tree structure.

FIG. 6 is a flow chart of analyzing a DNA chip by using GO according to a preferred embodiment of the present invention.

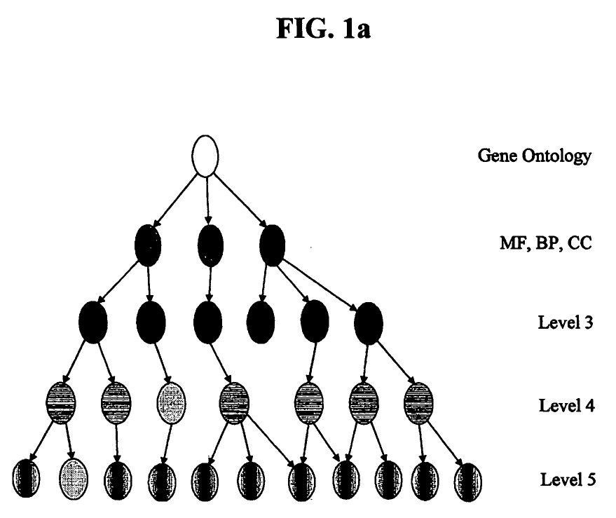

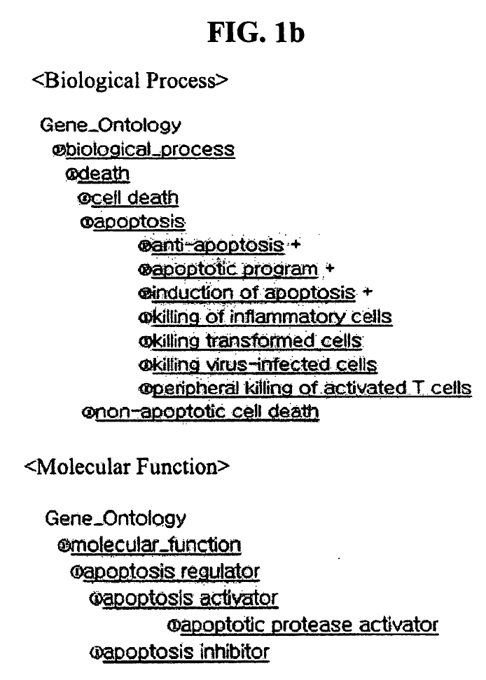

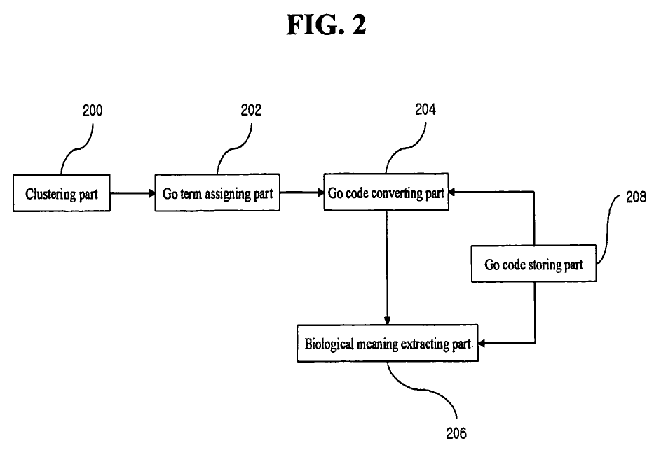

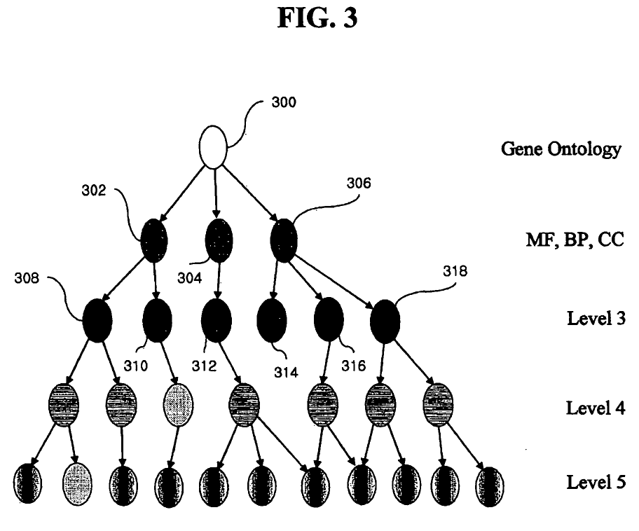

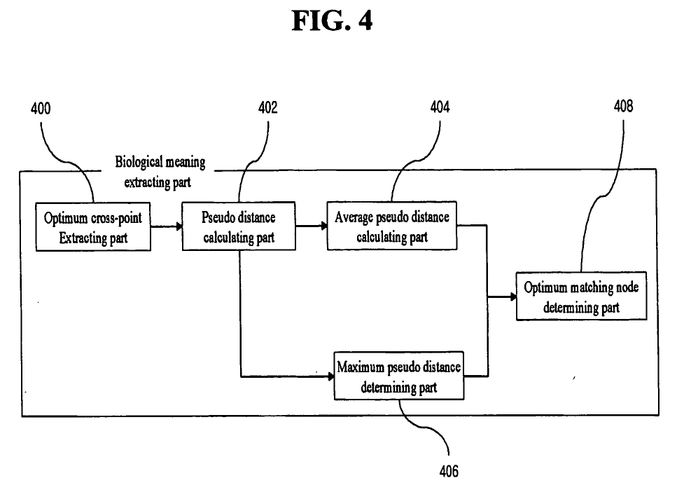

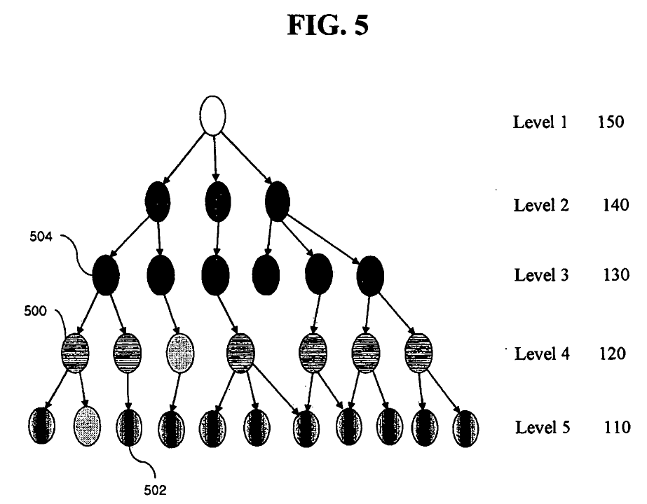

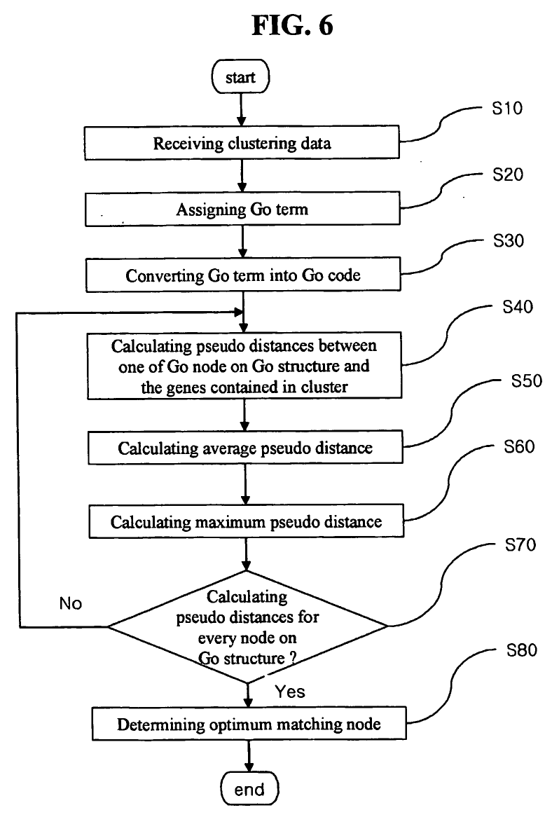

### INDUSTRIAL APPLICABILITY

According to the present invention, the biological analysis on the expression patterns of the genes obtained from the DNA chip can be performed systematically and automatically through the modeling of the GO hierarchical structure. Furthermore, the commonest and the most ideal the function of the genes contained in the cluster offered by statistical clustering of the data obtained from the DNA chip can be extracted by using the GO term and the GO tree structure.

Though the above embodiments have been described on the method for analyzing the DNA chip, it will be understood by those skilled in the art that the present invention may be applied to another bio chip such as a protein chip, and so on.

While the present invention has been particularly shown and described with reference to the above embodiments thereof, it will be understood by those skilled in the art that various changes in form and details may be effected therein without departing from the spirit and scope of the invention as defined by the appended claims.

What is claimed is:

1. A system for analyzing a bio chip comprising:
a GO(gene ontology) term assigning part for receiving a statistical clustering data obtained from the empirical results of the bio chip, and assigning relevant GO terms to every gene contained in each cluster;
a GO code converting part for converting the GO terms assigned by the GO term assigning part to the genes into GO codes, the GO code comprising a group of predetermined numbers; and
a biological meaning extracting part for calculating pseudo distances between one of GO terms contained in a predetermined group on GO tree structure and the GO terms corresponding to the genes contained in the cluster, and calculating at least one of average pseudo distance or maximum pseudo distance of the calculated pseudo distances, and calculating at least one of average pseudo distances or maximum pseudo distances for all GO terms included in the predetermined group on GO tree structure and the GO terms corresponding to the genes contained in the cluster, and determining an optimum GO term matching with the cluster.

2. The system according to claim 1, wherein the GO term assigning part assigns GO terms to the genes using biology database mining.

3. The system according to claim 1, wherein the GO code converting part coverts the GO terms into the GO codes according to a level of a GO term, a parent-node of the GO term and an order of the GO term in the level.

4. The system according to claim 1, wherein the biological meaning extracting part comprises:
an optimum cross-point extracting part for extracting optimum cross-points between the GO terms on the GO tree structure and the GO terms assigned to the genes contained in the predetermined group;
a pseudo distance calculating part for calculating pseudo distances between the GO terms on the GO tree structure and the GO terms assigned to the genes contained in the cluster by using the optimum cross-points information;
an average pseudo distance calculating part for calculating average pseudo distance of the pseudo distances calculated from the pseudo distance calculating part;
a maximum pseudo distance determining part for determining maximum distance among the pseudo distances calculated from the pseudo distance calculating part; and
an optimum matching node determining part for comparing average pseudo distances or maximum pseudo distances for all GO terms contained in the predetermined group, and determining a GO term with minimum value of the average pseudo distance or of the maximum pseudo distance to be optimum matching node of the cluster.

5. The system according to claim 4, wherein the GO terms contained in the predetermined group are all terms on the GO tree structure.

6. The system according to claim 4, wherein the GO terms contained in the predetermined group are GO terms included in a selected level on the GO tree structure.

7. The system according to claim 4, wherein the optimum cross-point extracting part determines a GO term in the lowest level among GO terms which include two GO terms in a lower level on the GO tree structure to be the optimum cross-point.

8. The system according to claim 1, wherein the GO tree structure comprises a level which a predetermined weight is granted to, and wherein the pseudo distance calculated by the pseudo distance calculating part is the weight granted to a level where the optimum cross-point exists.

9. A method for analyzing a bio chip comprising:
a) receiving a statistical clustering data obtained from empirical results of the bio chip to assign relevant GO terms to every gene contained in each cluster;
b) converting the GO terms assigned to the genes into GO codes, the GO code comprising a group of predetermined numbers;
c) calculating pseudo distances between one of GO terms contained in a predetermined group on GO tree structure and the GO terms corresponding to the genes contained in the cluster by using the GO codes;
d) calculating at least one of average pseudo distance or maximum pseudo distance of the pseudo distances calculated in the step (c); and
e) repeating the step (c) and the step (d) for every GO term on the GO tree structure contained in the predetermined group to determine an optimum GO term matching with the cluster.

10. The method according to claim 9, wherein the step (a) assigns GO terms to the genes using biology databases mining.

11. The method according to claim 9, wherein the step (b) coverts the GO terms into the GO codes according to a level of a GO term, a parent-node of the GO term and an order of the GO term in the level.

12. The method according to claim 9, wherein the GO terms contained in the predetermined group are all terms on the GO tree structure.

13. The method according to claim 9, wherein the GO terms contained in the predetermined group are GO terms included in a selected level on GO tree structure.

14. The method according to claim 9, wherein the step (c) comprises steps of:
extracting optimum cross-points between the GO terms on the GO tree structure and the GO terms assigned to the genes contained in the cluster; and
calculating pseudo distances between the GO terms on the GO tree structure and the GO terms assigned to the genes contained in the cluster by using the optimum cross-points information.

15. The method according to claim 9, wherein the step (e) determines a GO term on the GO tree structure with minimum value of the average pseudo distance or the maximum pseudo distance to be an optimum matching node of the cluster

16. The method according to claim 14, wherein the step for extracting the optimum cross-points determines a GO term in the lowest level among GO terms which include two GO terms in lower level on the GO tree structure to be the optimum cross-point.

17. The method according to claim 14, wherein the GO tree structure comprises a level which a predetermined weight is granted to, and wherein the calculated pseudo distance is an weight granted to a level where the optimum cross-point exists.

18. A digital device readable medium containing program instructions for executing an analysis of a bio chip, the medium comprising the program instructions for:
a) receiving a statistical clustering data obtained from empirical results of the bio chip, and for assigning relevant GO terms to every gene contained in each cluster;
b) converting the GO terms assigned to the genes into GO codes, the GO code comprising a group of predetermined numbers;
c) calculating pseudo distances between one of GO terms on GO tree structure contained a predetermined group and the GO terms corresponding to the genes contained in the cluster by using the GO codes;
d) calculating at least one of average pseudo distance or maximum pseudo distance of the pseudo distances calculated in the step (c); and
e) repeating the step (c) and the step (d) for every GO term on the GO tree structure contained in the predetermined group to determine an optimum GO term matching with the cluster.
